(19) United States
(12) Patent Application Publication
(10) Pub. No.: US 2008/0207750 A1
(43) Pub. Date: Aug. 28, 2008

(54) Use of N-Chlorotaurine for Treatment of Oozing Tissue Deficiencies

(75) Inventors: Waldemar Gottardi, Innsbruck (AT); Andreas Neher, Innsbruck (AT); Markus Nagl, Innsbruck (AT)

Correspondence Address: PROSKAUER ROSE LLP;PATENT DEPARTMENT, 1585 BROADWAY, NEW YORK, NY, 10036-8299, US

(73) Assignee: Pathogenics LLC, Cohasset, MA (US)

(21) Appl. No.: 11/912,368

(22) PCT Filed: Dec. 8, 2003
(86) PCT No.: PCT/US2003/039206

§ 371 Date: Oct. 23, 2007

Related U.S. Application Data
(60) Provisional application No. 60/431,615, filed on Dec. 6, 2002.

(51) Int. Cl. A61K 31/255; A61P 17/02

(52) U.S. Cl. 514/517

(57) ABSTRACT

A pharmaceutical composition containing N-chlorotaurine is administered to a mammal to treat oozing tissue deficiencies.

This application claims priority to U.S. provisional application 60/431,615, filed Dec. 6, 2002.

### BACKGROUND OF THE INVENTION

The present invention concerns the use of N-chlorotaurine (NCT) for the treatment of oozing tissue deficiencies and an NCT-containing drug for use in said treatment.

Oozing tissue deficiencies arise by a mechanically, physically or chemically induced impairment of the human body. This leads to damage to tissue provoking a visible secretion of fluid (oozing), which can be extended up to 5 days. The secreting fluid consists of blood plasma and leukocytes, which remove the destroyed parts of tissue.

Oozing tissue deficiencies can arise, for instance, subsequent to a tympanoplastic operation, and can cause impairment of incorporation of the substitute tympanic membrane, or its floating away caused by oozing. Following the tympanoplastic operation, the external auditory canal is tamponated with different kinds of packing for two to three weeks. After removal of the tamponade the external auditory canal is moist. This milieu predisposes to local infections triggered by moisture, mostly caused by bacteria and fungi. Dominant among them is Pseudomonas aeruginosa (Reference 13 at page 8).

The main aim in postoperative treatment must therefore be the fast desiccation of the external meatus as well as local disinfection to inhibit bacterial and fungal growth, without adverse side effects like pain, delay of epithelisation, or damage of the inner ear. Although tympanoplasty is a common surgical procedure in modern ear surgery, there exist only few publications (References 12-15 at pages 8-9) and no guidelines for postoperative treatment. Most of the recommendations are limited to postoperative packing of the outer ear canal with different materials (Reference 16 at page 9), which hardly addresses the infection problems. A peri- and postoperative prophylaxis with a systemic broad-spectrum antibiotic for 5 days is recommended by some authors (Id.). Unfortunately, Pseudomonas aeruginosa and fungi are usually not affected by such antibiotic therapy. Hütten et al. use the cauterant substance 10% AgNO3 as a local antiseptic for postoperative prophylaxis of infections (Id.).

A wide-spread method in former days was the irrigation with Castellani's solution, which has a strong antiseptic and drying effect (Reference 17 at page 9). However, the use of this substance has been prohibited in Germany since 1996, as it contains carcinogenic components (References 18 and 19 at page 9).

In addition, oozing after otoplastic operations has been treated until now, for example, by application of solutions of fuchsine. This therapy, however, turned out to be unsatisfactory and is deemed controversial because of possible carcinogenic properties of fuchsine.

Accordingly, there exists a need in the art for a new treatment for oozing tissue deficiencies.

Surprisingly, it was observed that NCT reduced the period of oozing subsequent to operations, e.g. otoplastic operations, and induced an accelerated healing process. Rinsing the ear channel with NCT solution provokes in general a drying of the ear channel and the tympanoplastic within 1-2 days.

It is known that NCT exerts microbicidal activity (References 1-8 at pages 7-8). An inhibitory action on the secretion of fluid in oozing tissue deficiencies has not been described until now.

The secretion of oozing tissue deficiencies contains granulocytes, which are known to produce and release NCT (References 9-11 at page 8). It is surprising that the amount of NCT released by granulocytes does not effect the desired drying, while an additional treatment with synthetic NCT stimulates the drying in such a way that oozing is completely stopped.

### SUMMARY OF THE INVENTION

The invention concerns the treatment of all oozing tissue deficiencies in humans and other mammals, e.g. wound areas caused by operations, tympanoplasty, burns, frostbites, skin injuries, ulcers, cauterization and tumours breaking through the skin surface.

For the treatment of oozing tissue deficiencies, NCT may be applied in the form of aqueous solutions at a concentration of 0.1% to 20%, preferably 0.5% to 2%. NCT can be used in combination with the usual pharmaceutical additives, e.g. thickeners, ointment bases, and stabilizers, with an effective concentration of 0.1% to 20% NCT in a preparation ready for use.

One aspect of the invention is a method for treating ulcers in a mammal comprising the topical administration to the ulcer of a pharmaceutical composition comprising an effective amount of NCT or pharmaceutically acceptable salt thereof.

Yet another aspect of the invention is a method for treatment of patients after ear surgery, comprising the administration to the ear canal of a pharmaceutical composition comprising an effective amount of NCT or pharmaceutically acceptable salts thereof.

### BRIEF DESCRIPTION OF THE DRAWINGS

FIG. 1 is a graph depicting the results of clinical scores of the outer ear canal during a Pilot Study of the use of NCT in postoperative care after ear surgery.

### DETAILED DESCRIPTION

It is intended that reference to NCT herein be interpreted to mean that the pharmaceutically acceptable salt may also be employed.

The terms “treat”, “treated”, “treating” or “treatment” are used herein to include preventative (e.g. prophylactic) and/or palliative treatment.

Mammals treated according to the invention include but are not limited to humans.

NCT, and aqueous solutions thereof, may be obtained, synthesized and purified by known methods (see, e.g., Reference 20 at page 9).

NCT may be administered as a topical treatment for all oozing tissue deficiencies known or to be discovered in humans and other mammals. Examples of oozing tissue deficiencies include but are not limited to: wound areas caused by operations; burns; frostbites; skin injuries; ulcers; cauterization; and tumours breaking through the skin surface. Preferably, the oozing tissue deficiencies include wound areas resulting from a tympanoplasty operation or a stapedotomy operation; ulcers with fibrous coatings (e.g. those resulting from burns); and ulcers with purulent coatings.

NCT is best used in a pure form, preferably as a crystalline sodium salt dissolved in sterile distilled water to a concentration of 0.1% to 20%, preferably 0.5% to 5%, more preferably 0.5% to 2%, and most preferably 1%. Purity may be verified by iodometric titration and spectrophotometry. The percentages specified in this application relate to percentage by weight of the total composition, unless otherwise specified.

The pharmaceutical composition comprising NCT may be in the form of an aqueous solution, as described above, or may take other forms, such as an ointment, cream, salve or paste with an effective concentration of 0.1% to 20% NCT in a preparation ready for use. By way of example, the following additives may be included in the pharmaceutical composition: thickeners, carriers, ointment bases, stabilizers and buffers.

Since aqueous solutions of NCT exhibit a pH of 8 and a broad spectrum activity against pathogens, they may advantageously be used without preservatives, buffers, or other common additives that might cause an allergic reaction in patients.

A method for the treatment of patients with chronic leg ulcers having purulent coatings by the twice-daily application of dressings soaked in a 1% aqueous solution of NCT is described in N. Nagl et al., “Therapeutics: Tolerability and efficacy of N-chlorotaurine in comparison with chloramine T for the treatment of chronic leg ulcers with a purulent coating: a randomized phase II study”, British Journal of Dermatology 2003; 149:590-597, the entire text of which is incorporated herein by reference.

The invention will be explained in more detail, with the aid of the following examples.

### EXAMPLE 1

### Tympanoplasty

A 46 year old human male patient with a traumatic perforation of the tympanic membrane was provided with a tympanoplasty. After removing incrustations and granulation tissue, the destroyed tympanic membrane was excised and substituted by a fascia temporalis. After taking away the tamponade, the microbiological culture of a swab from the reconstructed tympanic membrane and the ear channel was sterile. Subsequently, the oozing ear channel was treated one time daily for two days by instillation of 2 mL of aqueous 1% NCT solution. Already after one day oozing ceased and the incorporation of the temporal fascia proceeded without problems. The complete epithelization was achieved 3 days earlier than by the conventional treatment with fuchsine.

### EXAMPLE 2

### Tympanoplasty

A 54 year old human male patient suffering from a perforation of the tympanic membrane caused by a chronic inflammation of the middle ear received tympanoplasty. After removing incrustations and granulation tissue, the destroyed tympanic membrane was excised and substituted by a fascia temporalis. After taking away the tamponade, the microbiological culture of a swab from the reconstructed tympanic membrane and the ear channel was sterile. Subsequently, the oozing and swollen ear channel was treated one time daily for five days by instillation of 2 mL of aqueous 1% NCT solution. After two days, swelling and after three days oozing ceased and the incorporation of the temporal fascia proceeded without problems. The complete epithelization was achieved 3 days earlier than by the conventional treatment with fuchsine.

### EXAMPLE 3

### Leg Ulcer

A 30 year old human male patient suffering from an open leg ulcer with fibrous coating caused by a burn accident was treated two times daily with wound dressing soaked with aqueous 1% NCT solution. After a treatment of three days, the fibrous coating disappeared and the healing process went on without problems. Microbiological cultures of swabs before and after therapy were sterile.

### EXAMPLE 4

### Pilot Study (Tympanoplasty)

A pilot study was carried out to develop the establishment of a postoperative ear care regimen with the antiseptic, endogenous substance NCT.

Procedures: Local irrigations of the external auditory canal with 3 ml of 1% N-chlorotaurine solution were performed once daily until the canal was dry.

Reagents: Pure NCT as a crystalline sodium salt was dissolved in sterile distilled water to a concentration of 1% (55 mM). Purity was verified by iodometric titration and spectrophotometry. Since aqueous solutions of NCT exhibit a pH of 8 and a broad spectrum activity against pathogens, no preservatives and buffers were added. Solutions were stored at 2-4° C., where they are stable for 1 year, and allowed to reach room temperature before application to the patients.

Study Design: An open phase IIa study was performed in 12 patients after tympanoplasty. They received a single-shot antibiotic therapy with 2 g Spizef (cefotiam) i.v. perioperatively. The number of patients, their diagnosis, and the type of different surgical procedures are summarized in Table 1.

TABLE 1

| | | Number |
|---|---|---|
| Diagnosis | Operation | of cases |
| cholesteatoma | tympanoplasty type III according to | 3 |
| | Wullstein (Reference 12, page 8) | |
| otosclerosis | stapedotomy | 4 |
| chronic secretory | tympanoplasty type I according to | 1 |
| otitis media | Wullstein (Reference 12, page 8) | |
| chronic secretory | tympanoplasty type III according to | 1 |
| otitis media | Wullstein (Reference 12, page 8) | |
| tympanosclerosis | tympanoplasty type III according to | 3 |
| | Wullstein (Reference 12, page 8) | |

All subjects were treated with 1% N-chlorotaurine.

Study Protocol:

Subjects: The study population included 6 female and 6 male human patients, ranging in age from 32 to 66 years (median 48,1 years). Inclusion criteria was status post tympanoplasty. Exclusion criteria included topical treatment with other agents, systemic application of antibiotics or corticoids, pregnancy, and simultaneous participation in another study. No patients had to be withdrawn after inclusion.

Treatment and Time Course: After removing the ear-tamponade, which remained in place for 2 to 3 weeks postoperatively, 3 ml of the 1% aqueous NCT solution was applied once daily to the outer ear canal. The endpoint of the treatment was defined as a completely dry canal, corresponding to a clinical score of zero (see below for scoring).

Evaluation and Statistical Analysis: The primary criterion for the judgement of the status of the external meatus was a six-scale clinical score (0-5) based on visual observation via ear microscopy (zero=dry outer ear canal; 5=moist outer ear canal with inflammation).

The second criterion was subjective pain, ascertained by a visual analogue scale.

Clinical examination of the outer ear canal was performed daily for detection of moisture, signs of infection, and judgement of epithelisation of the neo-tympanon.

A completely dry external meatus (score zero) was considered as the endpoint criterion for the termination of the study.

Before and at the end of the study an audiogram was performed in all patients.

Moisture and inflammation of the external auditory canal during and after irrigation with NCT were compared to those before treatment using the Wilcoxon test. P-values<0.05 were considered to indicate statistical significance. Due to the limited number of patients no corrections for multiple comparisons were applied.

Results:

Efficacy of treatment:

On day one, one patient demonstrated a clinical score of 4, five patients a score of 3, and six a score of 2. No patient showed a score of 5.

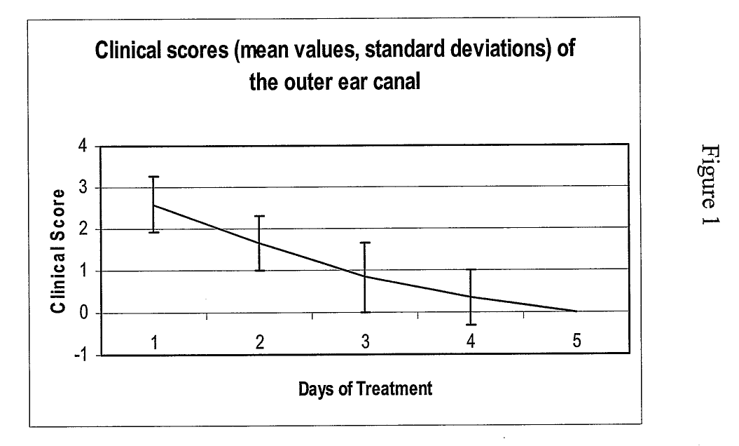

Rapid drying of the outer ear canal was detected in all patients. The average clinical score decreased daily (See FIG. 1). This decrease was highly statistically significant on all days with comparison to the baseline on day 1 (p-values compared with the Wilcoxon test: d2 vs. d1: p=0,001; d3 vs. d1: p=0,001; d4 vs. d1: p=0,002; d5 vs. d1: p=0,002).

No infection occurred in the postoperative course. The necessary time for achieving a score of zero was 2.75±0.87 days (mean±SD, range 2-5 days).

Epithelisation of the new tympanon found on the ear microscopy proceeded regularly without granulation. The time needed for complete epithelisation was 22.6±3.5 days (mean±SD, range 18-31 days).

Tolerability of treatment: The therapy was performed completely according to the study protocol and was tolerated very well by all patients without occurrence of any pain. All subjects completed the study. There were no signs of allergic reactions during the whole period of treatment. No dizziness or nystagmus occurred. There were no signs of damage to the inner ear, documented by an audiogram performed before and after treatment with NCT.

### REFERENCES

Each of the references cited below is incorporated by reference in its entirety herein.

1. Nagl M, Gruber A, Fuchs A et al. Impact of N-chlorotaurine on viability and production of secreted aspartyl proteinases of Candida spp. Antimicrob Agents Chemother 2002;46(6):1996.

2. Nagl M, Lass-Floerl C, Neher A, Gunkel A R, Gottardi W. Enhanced fungicidal activity of N-chlorotaurine in nasal secretion. J Antimicrob Chemother 2001;47(6):871.

3. Nagl M, Hess M, Pfaller K, Hengster P, Gottardi W. Bactericidal activity of micromolar N-chlorotaurine—evidence for its antimicrobial function in the human defense system. Antimicrob Agents Chemother 2000;44(9):2507.

4. Nagl M, Hengster P, Semenitz E, Gottardi W. The postantibiotic effect of N-chlorotaurine on Staphylococcus aureus. Application in the mouse peritonitis model. J Antimicrob Chemother 1999;43(6):805.

5. Nagl M, Gottardi W. Rapid killing of Mycobacterium terrae by N-chlorotaurine in presence of ammonium is caused by the reaction product monochloramine. J Pharm Pharmacol 1998;50(11):1317.

6. Nagl M, Larcher C, Gottardi W. Activity of N-chlorotaurine against herpes simplex- and adenoviruses. Antiviral Res 1998;38(1):25.

7. Nagl M, Gottardi W. Enhancement of the bactericidal efficacy of N-chlorotaurine by inflammation samples and selected N-H compounds. Hyg Med 1996;21597.

8. Nagl M, Gottardi W. In vitro experiments on the bactericidal action of N-chlorotaurine. Hyg Med 1992;17431.

9. Grisham M B, Jefferson M M, Melton D F, Thomas E L. Chlorination of endogenous amines by isolated neutrophils. J Biol Chem 1984;259(16):10404.

10. Passo S A, Weiss S J. Oxidative mechanisms utilized by human neutrophils to destroy Escherichia coli. Blood 1984;63(6):1361.

11. Test S T, Lampert M B, Ossanna P J, Thoene J G, Weiss S J. Generation of nitrogen-chlorine oxidants by human phagocytes. J Clin Invest 1984;74(4):1341.

12. Probst R, Grevers G, Iro H. Hals-Nasen-Ohren-Heilkunde. Thieme Verlag Stuttgart, New York, 2000, p. 246.

13. Kristiansen P. The diagnosis and management of malignant (necrotizing) otitis externa. J Am Acad Nurse Pract. 1999; 11:297-300.

14. Thessing J. HNO-Operationslehre, Begründet von Gerhard Theissing, mit Beiträgen von G. Rettinger, 3.vollständig überarbeitete und erweiterte Auflage; Thieme Verlag Stuttgart, New York 1996;344-366.

15. Duport N., San Jullian M, Allene M et al. Tympanoplasty. Postoperative care. Soins Chir. 1985;22-25.

16. Hutten R. Postoperative treatment in tympanoplasty. Acta Otorhinolaryngol Belg. 1977;31(6):608-14.

17. Saito T, Tanaka T, Tokuriki M et al. Recent outcome of tympanoplasty in the elderly. Otol Neurotol. 2001;22:153-157.

18. Wiesenthal A A, Garber L Z. New method for packing the external auditory canal, middle ear space, and mastoid cavities after otologic surgery. J Otolaryngol. 1999;28:260-265.

19. Schroer R, Krech T, Hommerich C P. Solutio Castellani-Untersuchungen und Bemerkungen zu einer altbewährten Tinktur. Dtsch.Ärzteb; 1990;87:C-2365-2366.

20. Gottardi W, Nagl M. Chemical properties of N-chlorotaurine soldium, a key compound in the human defense system. Arch Pharm (Weinheim). 2002;335:411-421.

1. A method of treating oozing tissue deficiencies in a mammal comprising the topical administration of a pharmaceutical composition comprising an effective amount of N-chlorotaurine or pharmaceutically acceptable salts thereof.

2. The method of claim 1, characterized in that the N-chlorotaurine is in aqueous solution at a concentration of 0.1% to 20%.

3. The method of claim 2, characterized in that the N-chlorotaurine is in aqueous solution at a concentration of 0.5% to 5%.

4. The method of claim 3, characterized in that the N-chlorotaurine is in aqueous solution at a concentration of 0.5% to 2%.

5. The method of claim 1, wherein the oozing tissue deficiency is selected from the group consisting of: wound areas caused by operations; burns; frostbites; skin injuries; ulcers; cauterization; and tumors breaking through the skin surface.

6. The method of claim 5, characterized in that the oozing tissue deficiency is a would area resulting from a tympanoplasty operation.

7. The method of claim 5, characterized in that the oozing tissue deficiency is a would area resulting from a stapedotomy operation.

8. The method of claim 5, characterized in that the oozing tissue deficiency is an ulcer with a fibrous coating.

9. The method of claim 5, characterized in that the oozing tissue deficiency is an ulcer with a purulent coating.

10. A method for the treatment of patients after ear surgery, comprising the administration to the ear canal of a pharmaceutical composition comprising an effective amount of N-chlorotaurine or pharmaceutically acceptable salts thereof.

11. The method of claim 10, characterized in that the N-chlorotaurine is in aqueous solution at the concentration of 0.1% to 20%.

12. The method of claim 11, characterized in that the N-chlorotaurine is in aqueous solution at a concentration of 0.5% to 5%.

13. The method of claim 12, characterized in that the N-chlorotaurine is in aqueous solution at a concentration of 0.5% to 2%

14. A method for the treatment of ulcers in a mammal comprising the topical administration to the ulcer of a pharmaceutical composition comprising an effective amount of N-chlorotaurine or pharmaceutically acceptable salts thereof.

15. The method of claim 14, wherein the N-chlorotaurine at a concentration of 0.1% to 20%.

16. The method of claim 15, characterized in that the N-chlorotaurine is in aqueous solution at a concentration of 0.5% to 5%.

17. The method of claim 16, characterized in that the N-chlorotaurine is in aqueous solution at a concentration of 0.5% to 2%.

18. The method of claim 15, wherein the ulcer is an ulcer with a fibrous coating.

19. The method of claim 15, wherein the ulcer is an ulcer with a purulent coating.

20. The method of claim 1, wherein the mammal or patient is a human.
